(19) United States
(12) Patent Application Publication
(10) Pub. No.: US 2011/0224762 A1
(43) Pub. Date: Sep. 15, 2011

(54) Therapeutic System for Securing a Hot or Cold Source to a Body

(76) Inventors: Thomas L. Gruber, Escondido, CA (US); Kathleen Finn, Escondido, CA (US)

(21) Appl. No.: 13/042,899

(22) Filed: Mar. 8, 2011

Related U.S. Application Data
(60) Provisional application No. 61/311,883, filed on Mar. 9, 2010.

(51) Int. Cl. A61F 7/08; A61F 7/10

(52) U.S. Cl. 607/112

(57) ABSTRACT

A therapeutic system for securing a hot or cold source to a body is disclosed herein. An elastic sleeve designed to enclose a portion of a body part includes one or more hook and loop fasteners. A hot or cold source with corresponding hook and loop fasteners is then secured to the one or more hook and loop fasteners of the elastic sleeve. A portion of the elastic sleeve is then folded back over itself and the hot or cold source to comfortably secure it to a desired body part.

### CROSS REFERENCE TO RELATED APPLICATIONS

This application is a non-provisional of U.S. Patent Application No. 61/311,883 filed on Mar. 9, 2010, the entirety of which is hereby incorporated by reference.

### STATEMENT REGARDING FEDERALLY SPONSORED RESEARCH OR DEVELOPMENT

This invention was not federally sponsored.

### BACKGROUND OF THE INVENTION

### Field of the Invention

This invention relates to the general field of therapeutic devices, and more specifically toward a therapeutic system for securing a hot or cold source to a body. An elastic sleeve designed to enclose a portion of a body part includes one or more hook and loop fasteners. A hot or cold source with corresponding hook and loop fasteners is then secured to the one or more hook and loop fasteners of the elastic sleeve. A portion of the elastic sleeve is then folded back over itself and the hot or cold source to comfortably secure it to a desired body part.

Various therapies require placing hot or cold sources near a part of the body. Cold packs are often used to reduce inflammation and swelling. Hot packs are often used to relax tightened or injured muscles or treat chronic conditions. Some injuries require successively applying cold and hot packs (collectively “temperature packs”) to an injured portion of the body to both reduce inflammation and relax muscles.

The prior art teaches many devices and methods for securing hot or cold packs to a portion of the body. One such device is an elastic sleeve with an associated pocket. The sleeve encloses the injured portion of the body. The hot or cold pack is removably secured within the pocket to apply the heat or cooling to the desired location of the body. However, this design only allows for temperature packs that match the size and shape of the pocket. Temperature packs that are larger than the pocket will not fit; temperature packs that are much smaller than the pocket will move around, fall out, and possibly not heat or cool the desired body part.

Another prior art device includes temperature packs that are sleeves themselves. The temperature pack is secured directly over the body part. This can lead to discomfort to the user since the heat or cold source comes in direct contact with the skin. Further, the same sleeve unit cannot be used to both heat up and cool down the desired location on the body. Other prior art methods include wrapping a cloth around the temperature pack and tying the cloth to the desired body part. Once again, direct contact of the temperature pack to the skin can be uncomfortable to the user and could damage tissue or nerve endings. Additionally, this method does not facilitate quick and easy removal and reapplication of a temperature pack.

Thus there has existed a long-felt need for a therapeutic system that secures a hot or cold source to a body and provides some layer of protection between the temperature pack and the body. Further there is a long-felt need for a therapeutic system that can easily be attached without assistance and allows for quick and easy removal and application. Additionally, there is a long-felt need for a therapeutic system that permits significant mobility during treatment.

### SUMMARY OF THE INVENTION

The current invention provides just such a solution by having a therapeutic system for securing a hot or cold source to a body. An elastic sleeve designed to enclose a portion of a body part includes one or more hook and loop fasteners. A hot or cold source with corresponding hook and loop fasteners is then secured to the one or more hook and loop fasteners of the elastic sleeve. A portion of the elastic sleeve is then folded back over itself and the hot or cold source is comfortably secured to a desired body part. The elastic sleeve extends the therapeutic treatment time and allows for significant mobility during treatment.

It is a principal object of the invention to provide a therapeutic system that can be used to successively apply hot and cold sources against a portion of a body.

It is another object of the invention to provide a therapeutic system that allows for the quick, efficient, and un-assisted removal and reapplication of a temperature pack.

It is a further object of this invention to provide a cost-effective therapeutic system for treating injuries that require cold packs, hot packs, or both.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

FIG. 1 is a top perspective view of the sleeve on the arm of a user according to one embodiment;

FIG. 2 is a top view thereof;

FIG. 3 is a side view thereof;

FIG. 4 is a top perspective view of the sleeve with a temperature pack;

FIG. 5 is a top perspective view of the sleeve folded over the temperature pack of FIG. 4; and

FIG. 6 is a top perspective view of the temperature pack.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

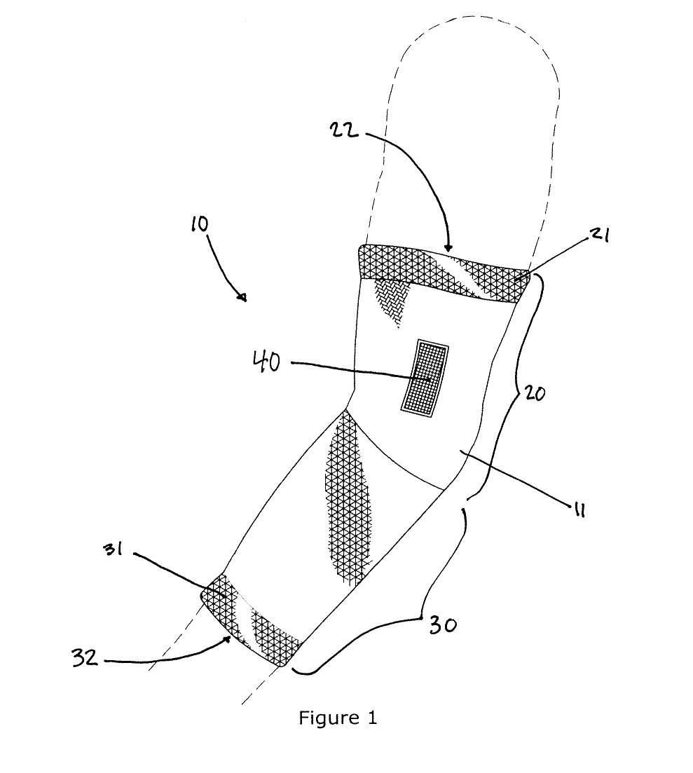

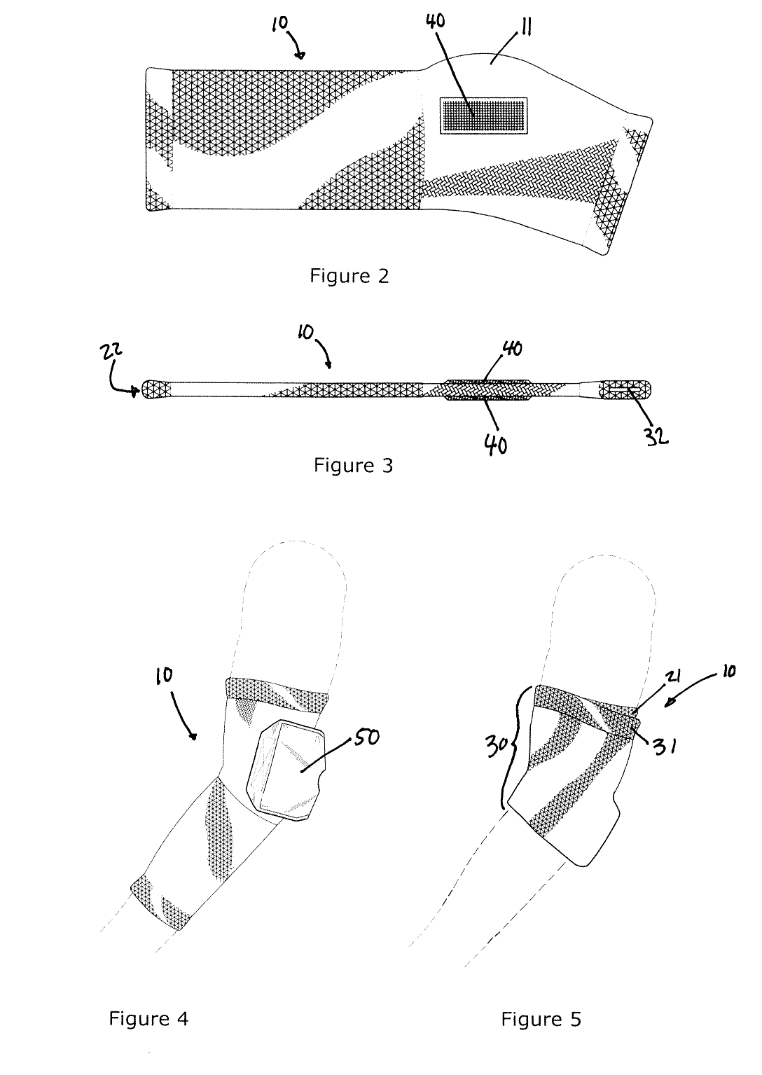

FIGS. 1, 2, and 3, illustrate a sleeve according to one embodiment of the current invention. A sleeve 10 has an upper region 20 and a lower region 30. The sleeve 10 has a semi-tubular shape, whereby there is an opening 32 in the lower portion and an opening 22 in the upper portion forming an inner cavity within the sleeve 10. At each end, there is an elastic band 31 and 21, where the elastic band has an elasticity greater than or equal to the elasticity of the remaining portion of the sleeve 10. Various materials can be used to fashion the sleeve, including acrylic, cotton, nylon, polyester, spandex, and other elastic materials.

The sleeve itself preferably, though not necessarily, forms an angle between the upper portion 20 and lower portion 30 and includes a bulge 11. The sleeve may be worn over a joint, such as over the elbow of an arm. Thus, the angle between the upper portion 20 and lower portion 30 more closely follows the natural angle of an appendage. The bulge 11 also allows for the joint, such as an elbow or knee, to rest therein and more comfortably secure the sleeve to the appendage. While the currently disclosed embodiment in the figures illustrates an angle and a bulge, it may be preferable to have no angle and no bulge. For example, if the current system is used over the forearm or lower back, no angle and bulge are preferred.

Hook and loop fasteners are used to initially secure a temperature pack to the sleeve. The hook and loop fasteners 40 are secured to the sleeve by means well known in the art, such as sewing, glue, and ultrasonic welding. The hook and loop fasteners may coincide with the bulge 11 in the lower region 30. However, it may also be preferable, depending on the application of the current system, to secure a temperature pack to a portion of the body away from a joint, thereby requiring the placement of the hook and loop fasteners 40 away from the bulge 11. As illustrated in FIG. 3, there are two hook and loop fasteners 40. While two hook and loop fasteners are preferable, more or less may be sufficient for particular applications of the current invention.

Temperature packs 50 can be sources of heat or cold. Hot packs transfer heat to the body while cold packs remove heat from the body. Temperature packs can be of various shapes and sizes with one or more hook and loop fasteners affixed thereto. The hook and loop fasteners of the temperature pack should be positioned such that they readily mate with the hook and loop fasteners 40 of the sleeve 10. In this manner, temperature packs are secured to the sleeve by means of hook and loop fasteners.

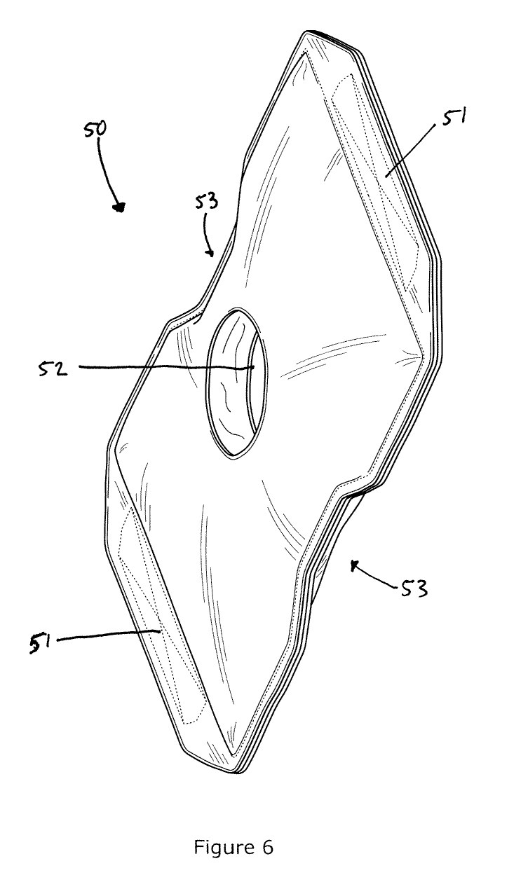

A preferred embodiment of the temperature pack 50 is shown in FIG. 6. The temperature pack 50 includes two ends 51 whereby hook and loop fasteners can be affixed. A hole 52 is included within the middle of the temperature pack. The hole 52 serves two purposes. First, it allows for the temperature pack 50 to comfortably fit around certain body parts such as the elbow. Second, it provides an area where no heat transfer occurs. This can be beneficial around body parts such as the elbow, which often do not require the application or removal of heat. Indentations 53 are also preferably included to allow the temperature pack 50 to more easily bend around portions of the body.

The system according to the current invention requires that the user wear a sleeve. The user takes the elastic sleeve and pulls it over a portion of the body. For example, a user may pull a sleeve 10 over the user's arm, whereby the upper portion 20 is pulled towards the shoulder. The bulge 11 fits over the user's elbow. The elasticity of the sleeve and, if applicable, the bulge in the sleeve helps secure the sleeve to the desired party of the body.

After placing the sleeve on the body, the temperature pack is affixed. As illustrated in FIG. 4, a temperature pack 50 is secured to the sleeve 10 over the hook and loop fasteners. Referring now to FIG. 5, the user then pulls the lower portion 30 of the sleeve 10 up over the temperature pack 50 and upper portion 20 of the sleeve. The elastic band 21 of the upper portion helps keep the sleeve in place; the elastic band 31 of the lower portion helps keep the lower portion 30 and sleeve 10 in place over the desired body part. To remove the temperature pack, the preceding steps may be performed in reverse.

As will become apparent to one skilled in the art, the temperature packs may be of different shapes and sizes. The hook and loop fasteners allow for the temperature pack to be quickly and easily secured to the sleeve without assistance by others. By folding the lower portion back up and over the upper portion, the temperature pack is further secured to the sleeve and therefore to the desired body part.

The system of the current invention may include a plurality of sleeves and a plurality of temperature packs, some cold and some hot. The plurality of sleeves may be individually designed for various parts of the body, including without limitation the arms, legs, torso, and neck. A plurality of temperature packs, both hot and cold, may be included. However, it is intended that there be no requirement for a one-to-one correlation between temperature packs and sleeves. In other words, the same temperature pack may be used on many different sleeves. To further this goal, it is preferable that the hook and loop fasteners have consistent relative dimensions, where these consistent relative dimensions match those of hook and loop fasteners on the temperature packs. For example, each temperature pack, whether hot or cold, may have two hook and loop fasteners that are two inches apart. These temperature packs may then be easily secured to sleeves that have corresponding hook and loop fasteners located two inches apart.

One of the key benefits of the current system and method is the ability to quickly and easily switch out temperature packs without assistance from others while keeping them properly secured to the body. For example, a particular injury may require an initial application of a cold pack, and then a further application of a hot pack. A sleeve according to the current invention is placed over the injured body part and a cold pack fastened thereto. The lower portion of the sleeve is then folded up and over the cold pack and upper portion of the sleeve to comfortably secure the cold pack to the injured body part. Then, after a period of time, the lower portion of the sleeve is pulled back, the cold pack is removed and a hot pack is fastened thereto. The lower portion of the sleeve is then once again folder up and over, but this time over the hot pack. These steps can be repeated multiple times as is required to treat the injured part of the body.

Because the lower portion is folded up and over the upper portion of the sleeve, various sized temperature packs can be used. The temperature packs are not required to be a specific shape to fit securely within a pocket; rather, temperature packs of different shapes and sizes can be used consecutively with the same sleeve. Further, the temperature pack itself is not adjacent to the skin; rather, there is a layer of material (the upper portion of the sleeve) that separates the two. Additionally, the lower portion of the sleeve when folded up and over the temperature pack acts to insulate the temperature pack. This insulating feature helps reduce heat loss to the environment for hot packs, and helps reduce heat transferred from the environment to cold packs thus extending the thermal application time.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

That which is claimed:

1. A therapeutic system comprising
a sleeve, where the sleeve comprises an upper portion and a lower portion, where the upper portion comprises one or more hook and loop fasteners, and
a temperature pack, where the temperature pack comprises hook and loop fasteners,
where the hook and loop fasteners of the temperature pack can mate with the hook and loop fasteners of the sleeve.

2. The therapeutic system of claim 1, wherein the temperature pack is a cold pack.

3. The therapeutic system of claim 1, wherein the temperature pack is a hot pack.

4. A method of applying heat or removing heat from a body part comprising the steps of:
placing an elastic sleeve over a body part of a user, where the elastic sleeve comprises an upper portion and a lower portion, where the upper portion comprises one or more hook and loop fasteners,
securing a temperature pack to the elastic sleeve, where the temperature pack comprises hook and loop fasteners, where the temperature pack is secured to the elastic sleeve by means of hook and loop fasteners, and
folding the lower portion of the elastic sleeve up and over the temperature pack.

5. The method of claim 4, further comprising the steps of:
pulling back the lower portion of the elastic sleeve, and
removing the temperature pack from the sleeve.

6. The method of claim 4, further comprising the steps of:
securing an additional temperature pack to the elastic sleeve, where the additional temperature pack comprises hook and loop fasteners, where the additional temperature pack is secured to the elastic sleeve by means of hook and loop fasteners, and
folding the lower portion of the elastic sleeve up and over the temperature pack.

7. The method of claim 6, wherein the temperature pack is a hot pack and the additional temperature pack is a cold pack.

8. The method of claim 6, wherein the temperature pack is a cold pack and the additional temperature pack is a hot pack.

9. A therapeutic system comprising
a plurality of sleeves, where the sleeve comprises an upper portion and a lower portion, where the upper portion comprises a means of securing a temperature pack thereto, and
a plurality of temperature packs, where each temperature pack comprises a corresponding means of securing the temperature pack to the upper portion of the sleeve.

10. The therapeutic system of claim 9, wherein the means of securing a temperature pack to the sleeve is hook and loop fasteners.
